(19) United States
(12) Patent Application Publication
(10) Pub. No.: US 2006/0047227 A1
(43) Pub. Date: Mar. 2, 2006

(54) System and method for colon wall extraction in the presence of tagged fecal matter or collapsed colon regions

(76) Inventor: Anna Jerebko, West Chester, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/176,533

(22) Filed: Jul. 7, 2005

Related U.S. Application Data
(60) Provisional application No. 60/604,106, filed on Aug. 24, 2004.

(51) Int. Cl. A61B 5/107

(52) U.S. Cl. 600/593; 128/898

(57) ABSTRACT

A system and method for extracting a colon wall are provided. The method comprises: placing seeds in an image of a patient abdomen; determining features of the seeds and voxels neighboring the seeds; and performing a region growing of the colon wall using a classifier trained to distinguish between the colon wall and nearby objects based on the features of the seeds and voxels neighboring the seeds.

### CROSS-REFERENCE TO RELATED APPLICATION

This application claims the benefit of U.S. Provisional Application No. 60/604,106, filed Aug. 24, 2004, a copy of which is herein incorporated by reference.

### BACKGROUND OF THE INVENTION

1. Technical Field

The present invention relates to medical image analysis, and more particularly, to a system and method for extracting a colon wall in the presence of tagged fecal matter or collapsed colon regions.

2. Discussion of the Related Art

Colon cancer currently ranks as the second leading cause of cancer-related deaths in the world. Most colorectal cancers arise from initially adenomatous polyps. Studies have shown that early detection and removal of colonic polyps can reduce the risk of colon cancer, thus decreasing the mortality rate. Unfortunately, conventional methods for the detection of colonic polyps are invasive, uncomfortable and have associated morbidity.

Computed tomography (CT) colonography or virtual colonoscopy has emerged as a potential alternative screening method for colonic polyps as well as masses. It combines helical CT scanning of the abdomen with visualization tools from non-invasive assessment of the colonic mucosa. However, the interpretation of virtual colonoscopy exams is time-consuming and the accuracy of polyp detection may depend on the display techniques utilized and the level of physician expertise.

Recently, computer-aided diagnosis and detection (CAD) systems have been developed to automatically detect polyps and masses and provide the location of suspicious regions of the colon. Such CAD systems tend to employ algorithms for polyp detection that take into account the transition between a colon wall 110 and air 120 (e.g., the black area) as shown in FIG. 1. Further, in the presence of fecal matter or stool 250 in a colon 200 as shown in FIG. 2, the algorithms must deal with the removal of the fecal matter 250 because a colon wall 210 to air 220 (e.g., the black area) transition is not present in areas covered by the fecal matter 250.

The removal, however, of fecal matter tends to result in artifacts that change the look and properties of the surface of the colon wall and affect the subsequent analysis and detection of polyps by both a human observer and a CAD algorithm. Accordingly, there is a need for a technique capable of compensating for the presence of fecal matter in a colon so that its presence does not adversely affect the analysis and detection of polyps therein.

In addition, when collapsed colon regions are present, parts of the colon become virtually disconnected and it is difficult to trace the colon wall or its centerline in the collapsed regions. Accordingly, there is a need for a technique capable of analyzing a colon wall in the presence of collapsed regions, that improves the sensitivity of existing CAD algorithms, and that enhances the quality of virtual colonoscopy “fly-through” and centerline extraction techniques.

### SUMMARY OF THE INVENTION

The present invention overcomes the foregoing and other problems encountered in the known teachings by providing a system and method for extracting a colon wall in the presence of tagged fecal matter or collapsed colon regions that aids in the diagnosis and detection of diseases associated with the colon.

In one embodiment of the present invention, a method for extracting a colon wall, comprises: placing seeds in an image of a colon; determining features of the seed voxels and voxels neighboring the seeds; and performing a region growing of the colon wall using a classifier trained to distinguish between the colon wall and nearby objects based on the features of the seeds and voxels neighboring the seeds.

The seeds are placed in one of the colon wall, in air near the colon wall, in fat near the colon wall, in fecal matter near the colon wall or in a collapsed region of the colon wall. The seeds are placed automatically or manually. The image of the patient abdomen is acquired using one of a CT or magnetic resonance (MR) imaging technique.

The features are one of statistical properties of intensity, shape, texture or distance features of the seeds and voxels neighboring the seeds. The statistical properties are one of minimum, maximum or moments. The nearby objects are one of fecal matter, air, muscle, fat or liquid.

The method further comprises: acquiring image data from a patient; selecting sample voxels from the image data; determining features of the sample voxels and voxels neighboring the sample voxels; training a classifier to distinguish between the colon wall and nearby objects; and validating the classifier. The method also comprises restricting the region growing from leaking into adjacent regions.

In another embodiment of the present invention, a method for tracking a colon wall, comprises: placing a plurality of seed voxels in an image of a patient abdomen; determining features of the seed voxels and their neighboring voxels, wherein the features are one of statistical properties of intensity, shape, texture or distance features of the seed voxels and their neighboring voxels; and determining a connectivity of the colon wall by performing a region growing of the colon wall using a classifier trained to distinguish between the colon wall and nearby objects based on the features.

The statistical properties are one of minimum, maximum or moments. The nearby objects are one of fecal matter, air, muscle, fat or liquid.

In yet another embodiment of the present invention, a system for extracting a colon wall, comprises: a memory device for storing a program; a processor in communication with the memory device, the processor operative with the program to: place a seed in an image of a patient abdomen; determine features of the seed and voxels neighboring the seed; and perform a region growing of the colon wall using a classifier trained to distinguish between the colon wall and nearby objects based on the features of the seed and voxels neighboring the seed.

The seed is placed in one of the colon wall, in air inside the colon, in fat near the colon wall, in fecal matter or in a collapsed region of the colon wall. The features are one of statistical properties of intensity, shape, texture or distance of the seed and voxels neighboring the seed. The statistical properties are one of minimum, maximum or moments. The nearby objects are one of fecal matter, air, muscle, fat or liquid.

The processor is further operative with the program code to: acquire image data from a patient; select a sample voxel from the image data; determine features of the sample voxel and voxels neighboring the sample voxel; train a classifier to distinguish between the colon wall and nearby objects; and validate the classifier. The processor is also operative with the program code to restrict the region growing from leaking. The image of the patient abdomen is acquired using one of a CT or MR imaging device.

In another embodiment of the present invention, a method for locating polyps in a colon is provided. The method comprises: placing seeds in an image of a colon; determining features of the seeds and voxels neighboring the seeds; extracting a wall of the colon by performing a region growing of the colon wall using a classifier trained to distinguish between the colon wall and nearby objects based on the features of the seeds and voxels neighboring the seeds; and locating polyps on the colon wall using the extracted colon wall.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 is an image of a colon without fecal matter;

FIG. 2 is an image of a colon with tagged fecal matter;

FIG. 3 is a block diagram of a system for extracting a colon wall according to an exemplary embodiment of the present invention;

FIG. 4 is a flowchart illustrating a method for training a classifier to distinguish a colon wall from nearby objects according to an exemplary embodiment of the present invention;

FIG. 5 is a flowchart illustrating a method for extracting a colon wall according to an exemplary embodiment of the present invention; and

FIG. 6 is an image of a colon with non-uniformly tagged fecal matter.

### DETAILED DESCRIPTION OF EXEMPLARY EMBODIMENTS

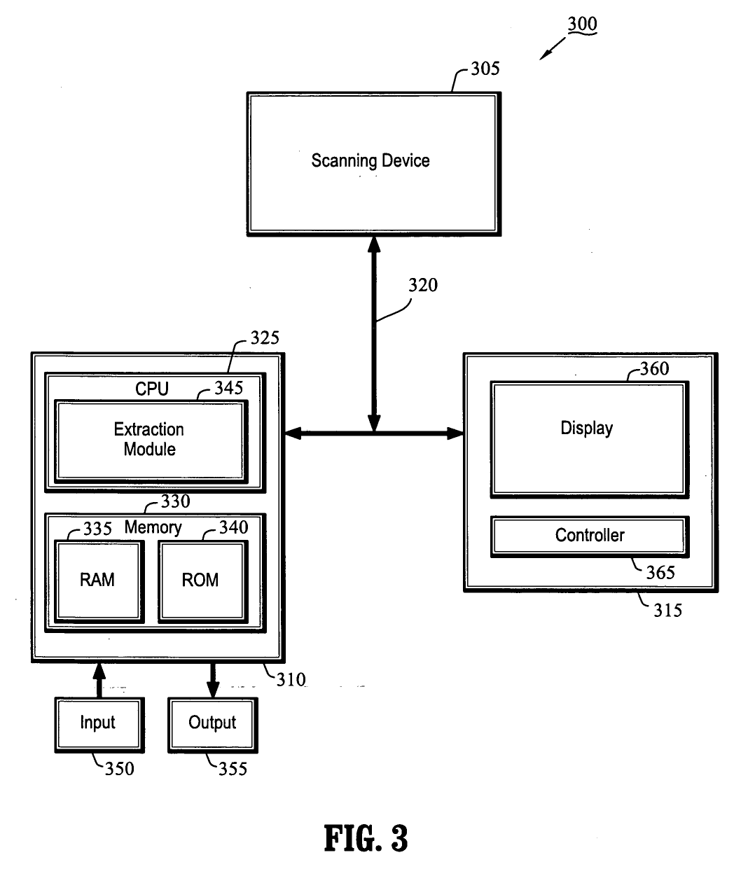

FIG. 3 is a block diagram of a system 300 for extracting a colon wall in the presence of tagged fecal matter or collapsed colon regions according to an exemplary embodiment of the present invention. As shown in FIG. 3, the system 300 includes, inter alia, a scanning device 305, a personal computer (PC) 310 and an operator's console 315 connected over, for example, an Ethernet network 320. The scanning device 305 may be an MR imaging device, a CT imaging device, a helical CT imaging device or a hybrid imaging device capable of CT, MR, positron emission tomography (PET) or other imaging techniques.

The PC 310, which may be a portable or laptop computer, a workstation, etc., includes a central processing unit (CPU) 325 and a memory 330, which are connected to an input 350 and an output 355. The CPU 325 includes an extraction module 345 that includes one or more methods for extracting a colon wall in the presence of tagged fecal matter or collapsed colon regions.

The memory 330 includes a random access memory (RAM) 335 and a read only memory (ROM) 340. The memory 330 can also include a database, disk drive, tape drive, etc., or a combination thereof. The RAM 335 functions as a data memory that stores data used during execution of a program in the CPU 325 and is used as a work area. The ROM 340 functions as a program memory for storing a program executed in the CPU 325. The input 350 is constituted by a keyboard, mouse, etc., and the output 355 is constituted by a liquid crystal display (LCD), cathode ray tube (CRT) display, or printer.

The operation of the system 300 is controlled from the operator's console 315, which includes a controller 365, for example, a keyboard, and a display 360, for example, a CRT display. The operator's console 315 communicates with the PC 310 and the scanning device 305 so that two-dimensional (2D) image data collected by the scanning device 305 can be rendered into three-dimensional (3D) data by the PC 310 and viewed on the display 360. It is to be understood that the PC 310 can be configured to operate and display information provided by the scanning device 305 absent the operator's console 315, using, for example, the input 350 and output 355 devices to execute certain tasks performed by the controller 365 and display 360.

The operator's console 315 may further include any suitable image rendering system/tool/application that can process digital image data of an acquired image dataset (or portion thereof) to generate and display 2D and/or 3D images on the display 360. More specifically, the image rendering system may be an application that provides 2D/3D rendering and visualization of medical image data, and which executes on a general purpose or specific computer workstation. Moreover, the image rendering system may enable a user to navigate through a 3D image or a plurality of 2D image slices. The PC 310 may also include an image rendering system/tool/application for processing digital image data of an acquired image dataset to generate and display 2D and/or 3D images.

As shown in FIG. 3, the extraction module 345 may also be used by the PC 310 to receive and process digital medical image data, which as noted above, may be in the form of raw image data, 2D reconstructed data (e.g., axial slices), or 3D reconstructed data such as volumetric image data or multiplanar reformats, or any combination of such formats. The data processing results can be output from the PC 310 via the network 320 to an image rendering system in the operator's console 315 for generating 2D and/or 3D renderings of image data in accordance with the data processing results, such as segmentation of organs or anatomical structures, color or intensity variations, and so forth.

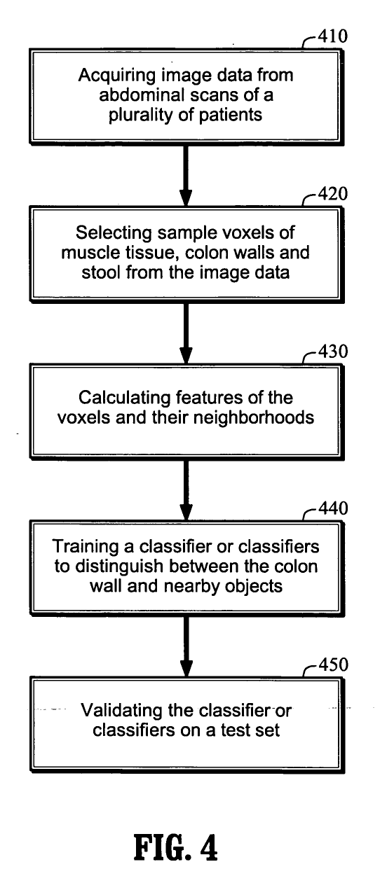

FIG. 4 illustrates a method for training a classifier to distinguish a colon wall from nearby objects. As shown in FIG. 4, image data from an abdominal scan or scans of a patient or patients is acquired (410). This is accomplished by using the scanning device 305, in this example a CT scanner, which is operated at the operator's console 315, to scan a patient's abdomen thereby generating a series of 2D image slices associated with a colon. The 2D image slices are then combined to form a 3D image.

After the CT image data is acquired, data samples of tagged and un-tagged fecal matter or stool, fat or muscle near the colon wall, the colon wall itself, collapsed colon regions, air, water or contrast matter are selected (420). Next, features such as statistical properties of voxels of individual sample points and the areas (e.g., neighborhoods) surrounding each of the voxels are calculated (430). The statistical properties that are calculated may be, for example, minimum, maximum and the moments of intensity (such as standard deviation, skewness and kurtosis). It is to be understood that the size of the neighborhoods could vary and can be determined based on a number of factors such as the thickness of the colon wall, collapsed colon regions, air, stool, fat or muscle being sampled.

Additional features characterizing shape, texture, distance, and statistical properties of local neighborhoods of different sizes around the sample points may be calculated in step 430. A wide variety of feature selection algorithms such as greedy search or genetic algorithms can be used to select relevant features and neighborhood sizes to be used in a subsequent classifier training technique.

Using the calculated features and statistical properties of the sample points and their local neighborhoods, a classifier or multiple classifiers are then trained to distinguish between the colon wall and nearby objects such as fat, muscle, air, stool or fluid inside the colon (440). It is to be understood that classifier training techniques employing semi-supervised, un-supervised of fully-supervised multi- or one-class classifications can be used in this step. Once the classifier or classifiers have been trained to distinguish between the colon wall and nearby objects, a validation such as a leave-one-out or N-fold cross-validation technique is performed, as well as a validation on an independent sequestered test set (450).

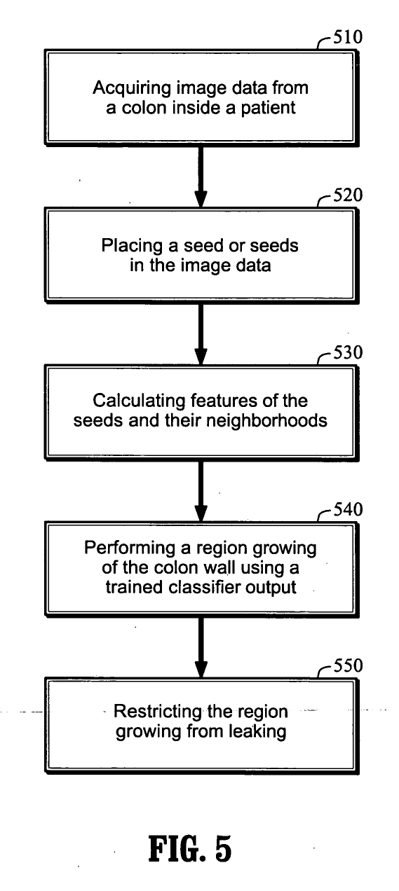

FIG. 5 is a flowchart showing an operation of a method for extracting a wall from a colon in the presence of tagged fecal matter or collapsed colon regions according to an exemplary embodiment of the present invention. As shown in FIG. 5, image data is acquired from, for example, an abdominal CT scan of a patient (510). This is accomplished by using the same or similar techniques described above with reference to step 410.

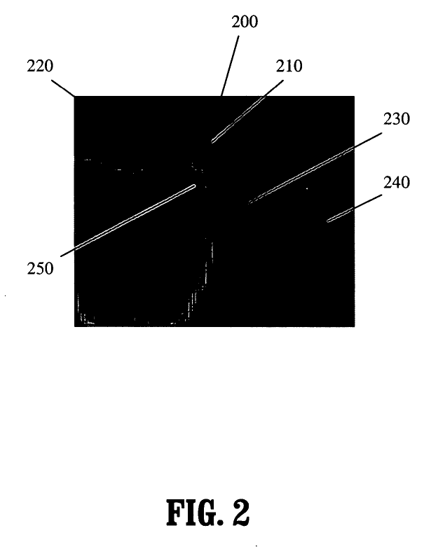

After the CT image data is acquired from the colon, a seed or seeds are placed in or around the colon (520). For example, the seeds may be placed in the colon wall 210 or in air pockets 220 inside the colon 200 as shown in FIG. 2. In addition, the seeds may be placed in the fat 240 or muscle 230, in the fecal matter 250 or in collapsed colon regions. The fecal matter 250 may be tagged by having a patient ingest an oral contrast agent such as barium or iodine that causes the fecal matter 250 or stool to have a distinctive coloring.

It is to be understood that in step 520 the seeds could be placed automatically by using an algorithm that determines the location of air pockets in a colon or stool in the colon and locates the colon wall. The seeds may also be placed manually. For example, a user may simply click on a desired seed point in or around a colon using a mouse cursor.

Once the seeds have been placed, features characterizing the shape, texture, distance and statistical properties of the seeds and local neighborhoods are calculated (530). This is accomplished by using the same or similar techniques described above with reference to step 430. The classifier or classifiers that were trained in step 440 are then applied together with a region growing of the colon wall 210 (540). More specifically, a region growing of the colon wall 210 is performed using the trained classifier or classifier's output and proximity and similarity measurements for all voxels. Upon completing the region growing, the connectivity of the colon wall 210 is determined, thus enabling the colon wall 210 to be extracted, traced or tracked. It is to be understood that in addition to applying the classifier or classifiers in the process of the region growing in step 540, other similarity measures may also be applied.

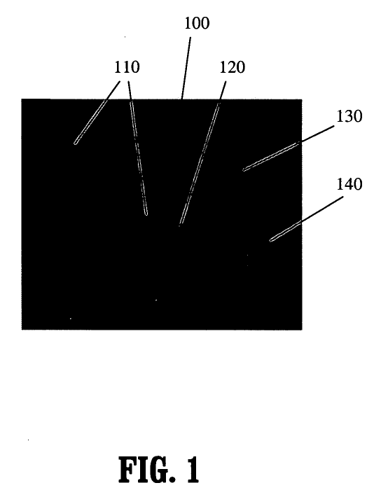

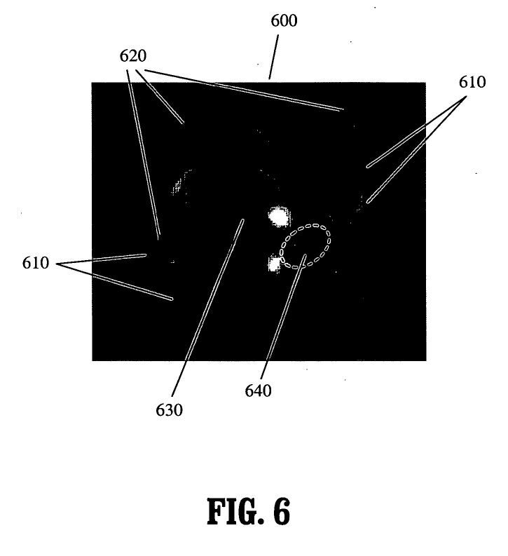

To further enhance the connectivity of a region grown colon wall and thus provide a medical practitioner or a polyp detection algorithm with a clearer and artifact free image of the colon wall for analysis, a set of post-processing steps may be performed on the region grown colon wall (550). One such process involves restricting the region growing from leaking into an un-tagged portion 640 of fecal matter 630 where there is no contrast agent 620. An example of this is shown in a colon image 600 of FIG. 6. For example, by removing voxels that have less than a predefined number of neighbors, applying morphological operations or by removing small detached clusters, a region growing can be prevented from leaking into the un-tagged portion 640 of fecal matter 630. Further, although care should be taken in regions where muscle 130 or fat 140 is close to the colon wall 110 as shown in FIG. 1, segmentation leakage into the muscle 130 does not affect the quality of the region growing inside the colon wall 110 that is of importance to clinicians, surface and volume rendering techniques and polyp detection algorithms in most instances.

In accordance with an exemplary embodiment of the present invention, a colon wall may be extracted as a thin muscle layer in the presence of tagged or partially tagged fecal matter or collapsed colon regions. Thus, enabling the inner portion of the colon wall to be visualized for a “fly-through” during a virtual colonoscopy, used for local endoscopic views of polyps located thereon or used in conjunction as an extension for or an alternative to manual or automated computer-aided diagnosis and polyp detection techniques.

It is to be further understood that because some of the constituent system components and method steps depicted in the accompanying figures may be implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be implemented without departing from the spirit and scope of the present invention.

It is therefore intended, that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

What is claimed is:

1. A method for extracting a colon wall, comprising:
placing seeds in an image of a patient abdomen;
determining features of the seeds and voxels neighboring the seeds; and
performing a region growing of the colon wall using a classifier trained to distinguish between the colon wall and nearby objects based on the features of the seeds and voxels neighboring the seeds.

2. The method of claim 1, wherein the seeds are placed in one of the colon wall, in air inside the colon, in fat near the colon wall, in fecal matter inside the colon or in a collapsed region of the colon wall.

3. The method of claim 2, wherein the seeds are placed automatically or manually.

4. The method of claim 2, wherein the fecal matter is tagged.

5. The method of claim 1, wherein the image of the patient abdomen is acquired using one of a CT or MR imaging technique.

6. The method of claim 1, wherein the features are one of statistical properties of intensity, shape, texture or distance features of the seeds and voxels neighboring the seeds.

7. The method of claim 6, wherein the statistical properties are one of minimum, maximum or moments.

8. The method of claim 1, wherein the nearby objects are one of fecal matter, air, muscle, fat or liquid.

9. The method of claim 1, further comprising:
acquiring image data of an abdominal scan of a patient;
selecting sample voxels from the image data;
determining features of the sample voxels and voxels neighboring the sample voxels;
training a classifier to distinguish between the colon wall and nearby objects; and
validating the classifier.

10. The method of claim 1, further comprising:
restricting the region growing from leaking.

11. A method for tracking a colon wall, comprising:
placing a plurality of seed voxels in an image of a colon;
determining features of the seed voxels and their neighboring voxels, wherein the features are one of statistical properties of intensity, shape, texture or distance features of the seed voxels and their neighboring voxels; and
determining a connectivity of the colon wall by performing a region growing of the colon wall using a classifier trained to distinguish between the colon wall and nearby objects based on the features.

12. The method of claim 11, wherein the statistical properties are one of minimum, maximum or moments.

13. The method of claim 11, wherein the nearby objects are one of fecal matter, air, muscle, fat or liquid.

14. A system for extracting a colon wall, comprising:
a memory device for storing a program;
a processor in communication with the memory device, the processor operative with the program to:
place a seed in an image of a patient abdomen;
determine features of the seed and voxels neighboring the seed; and
perform a region growing of the colon wall using a classifier trained to distinguish between the colon wall and nearby objects based on the features of the seed and voxels neighboring the seed.

15. The system of claim 14, wherein the seed is placed in one of the colon wall, in air inside the colon, in fat near the colon wall, in fecal matter or in a collapsed region of the colon wall.

16. The system of claim 14, wherein the features are one of statistical properties, shape, texture or distance of the seed and voxels neighboring the seed.

17. The system of claim 16, wherein the statistical properties are one of minimum, maximum or moments.

18. The system of claim 14, wherein the nearby objects are one of fecal matter, air, muscle, fat or liquid.

19. The system of claim 14, wherein the processor is further operative with the program code to:
acquire image data of a patient abdomen;
select a sample voxel from the image data;
determine features of the sample voxel and voxels neighboring the sample voxel;
train a classifier to distinguish between the colon wall and nearby objects; and
validate the classifier.

20. The system of claim 14, wherein the processor is further operative with the program code to:
restrict the region growing from leaking.

21. The system of claim 14, wherein the image of the patient abdomen is acquired using one of a CT or MR imaging device.

22. A method for locating polyps in a colon, comprising:
placing seeds in an image of a colon;
determining features of the seeds and voxels neighboring the seeds;
extracting a wall of the colon by performing a region growing of the colon wall using a classifier trained to distinguish between the colon wall and nearby objects based on the features of the seeds and voxels neighboring the seeds; and
locating polyps on the colon wall using the extracted colon wall.
